(19) United States
(12) Patent Application Publication
(10) Pub. No.: US 2025/0313589 A1
(43) Pub. Date: Oct. 9, 2025

(54) METHODS OF MODIFYING METHYLCYTOSINE OR DERIVATIVE THEREOF USING A PHOTOREDOX REACTION, AND METHODS OF USING THE SAME TO DETECT THE METHYLCYTOSINE OR DERIVATIVE THEREOF IN A POLYNUCLEOTIDE

(71) Applicant: Illumina, Inc., San Diego, CA (US)

(72) Inventors: Alexandre Hofer, Cambridge (GB); Elena Cressina, Cambridge (GB)

(73) Assignee: Illumina, Inc., San Diego, CA (US)

(21) Appl. No.: 18/867,393

(22) PCT Filed: Jan. 31, 2024
(86) PCT No.: PCT/US2024/013857

§ 371 Date: Nov. 19, 2024

Related U.S. Application Data
(60) Provisional application No. 63/482,742, filed on Feb. 1, 2023.

(51) Int. Cl. C07H 21/00; C12Q 1/6876

(52) U.S. Cl. CPC C07H 21/00; C12Q 1/6876; C12Q 2600/154

(57) ABSTRACT

Disclosed herein are methods of using photoredox reactions to modify 5-methylcytosine (5-mC), 5-hydroxymethylcytosine (5-hmC), 5-formlcytosine (5-fC), or 5-carboxylcytosine (5-caC) in a polynucleotide. In some examples, a photoredox reaction is used to install a functional group at the 5-position of the 5-caC, wherein the installed functional group further reacts with the 5-caC to form a product having at least two rings. In other examples, a photoredox reaction is used to install a functional group at the 5-methyl group of the 5-mC, wherein the installed functional group further reacts with the 5-mC to form a product having at least two rings. In other examples, a photoredox reaction is used to oxidize the 5-mC or 5-hmC to 5-fC; and a functional group at the 5-position of the 5-fC, wherein the installed functional group further reacts with the 5-fC to form a product having at least two rings.

### CROSS-REFERENCE TO RELATED APPLICATIONS

This application claims the benefit of U.S. Provisional Patent Application No. 63/482,742, filed on Feb. 1, 2023 and entitled “Methods of Modifying Methylcytosine or Derivative Thereof Using a Photoredox Reaction, and Methods of Using the Same to Detect the Methylcytosine or Derivative Thereof in a Polynucleotide,” the entire contents of which are incorporated by reference herein.

### FIELD

This application relates to modifying methylcytosine, and using the modified methylcytosine to detect the methylcytosine in a polynucleotide.

### SEQUENCE LISTING

The instant application contains a Sequence Listing XML which has been submitted electronically in XML format and is hereby incorporated by reference in its entirety. The Sequence Listing XML is named IP-2468-PCT.xml, is about 24 KB in size, and has a date of creation of Jan. 31, 2024.

### BACKGROUND

Within living organisms, such as humans, selected cytosines in the genome may become methylated. A common method used to detect methylated cytosines is sodium bisulfite sequencing. One issue with this method is that it often results in greater than 95% of the input DNA being degraded. Borane-containing compounds can be used in various protocols to detect methylated cytosines. However, previously known boranes can also degrade DNA. Thus, new methods and compositions are needed to detect methylated DNA that reduces DNA degradation.

### SUMMARY

Examples provided herein are related to methods of modifying methylcytosine or a derivative thereof using a photoredox reaction, and methods of using the same to detect the methylcytosine or derivative thereof in a polynucleotide.

Some examples herein provide a method of modifying 5-carboxylcytosine (5-caC) in a polynucleotide. The method may include using a photoredox reaction to install a functional group at the 5-position of the 5-caC. The installed functional group may further react with the 5-caC to form a product having at least two rings.

In some examples, the product has the structure:

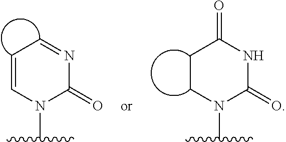 [SMILES: *n1cccnc1=O ; *N1CCC(=O)NC1=O]

In some examples, the photoredox reaction includes a photoinduced decarboxylative radical reaction replacing the 5-carboxyl group with the functional group R₀:

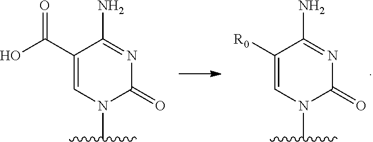

In some examples, the photoinduced decarboxylative radical reaction uses

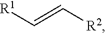

a photocatalyst or photosensitizer, and light. In some examples, R¹ includes an electron withdrawing group. In some examples, the electron withdrawing group is selected from the group consisting of aldehyde, ketone, ester, amide, hydrazido, cyano, and nitro. In some examples, R² promotes formation of at least one of the two rings.

In some examples, R¹ is an ester, and R² is H. Illustratively,

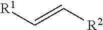

can be

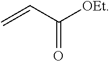

The photoredox reaction may form an intermediate:

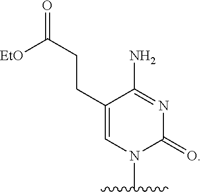 [SMILES: *n1cc(CCC(=O)OCC)c(N)nc1=O]

The intermediate may rearrange to form the product including:

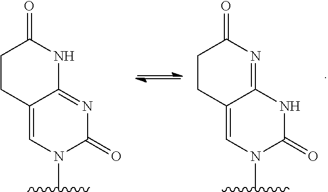 [SMILES: *n1cc2c(nc1=O)NC(=O)CC2 ; *N1C=C2CCC(=O)N=C2NC1=O]

In some examples, R¹ is an amide, and R² is ethanethiol. Illustratively,

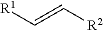

may be

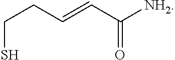

The photoredox reaction may form an intermediate:

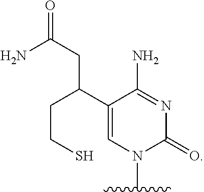 [SMILES: *n1cc(C(CCS)CC(N)=O)c(N)nc1=O]

The intermediate may rearrange to form the product including:

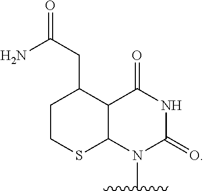 [SMILES: *N1C(=O)NC(=O)C2C(CC(N)=O)CCSC21]

In some examples,

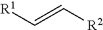

is diethyl ethylidenemalonate. The photoredox reaction may form an intermediate:

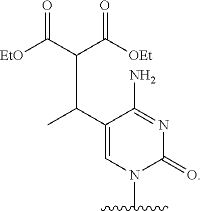 [SMILES: *n1cc(C(C)C(C(=O)OCC)C(=O)OCC)c(N)nc1=O]

The intermediate may rearrange to form the product including:

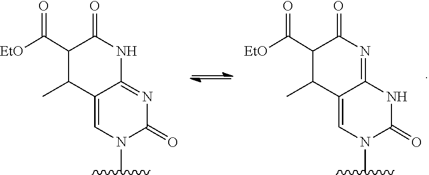 [SMILES: *n1cc2c(nc1=O)NC(=O)C(C(=O)OCC)C2C ; *N1C=C2C(=NC(=O)C(C(=O)OCC)C2C)NC1=O]

In some examples, the reaction further uses a donor-acceptor complex. In some examples, the donor may be selected from the group consisting of: biphenyl and phenanthrene. In some examples, the acceptor is selected from the group consisting of: 1,4-dicyanonaphthalene, 9,10-dicyanoanthracene, and 1,4-dicyanobenzene.

In some examples, the reaction further uses a copper salt and an oxidant. In some examples, the copper salt includes a Cu(I) salt or a Cu(II) salt. In some examples, the Cu(I) salt is selected from the group consisting of: CuBr, Cu(Oac), and Cu(Otf), or wherein the Cu(II) salt is selected from the group consisting of: CuBr₂, Cu(Oac)₂, and Cu(Otf)₂. In some examples, the oxidant is selected from the group consisting of N-fluorobenzene sulfonamide, Selectfluor, 1-fluoropyridinium or derivative thereof, and dicumyl peroxide.

In some examples, the photoredox reaction includes a photoinduced deoxygenative radical reaction functionalizing the 5-carboxyl group to include a functionalized 5-acyl group —(CO)R₀:

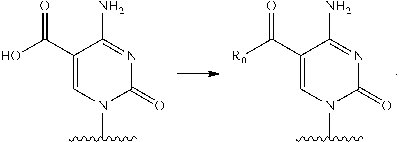

In some examples, the photoredox reaction uses

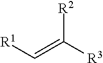

and light. In some examples, R¹ is an electron withdrawing group or includes an aryl group. In some examples, the electron withdrawing group is selected from the group consisting of aldehyde, ketone, ester, amide, hydrazido, cyano, and nitro. In some examples, R² and R³ promote formation of at least one of the two rings.

In some examples,

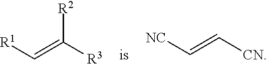

The photoredox reaction may form an intermediate:

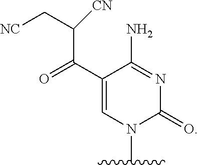 [SMILES: *n1cc(C(=O)C(C#N)CC#N)c(N)nc1=O]

The intermediate may rearrange to form the product including:

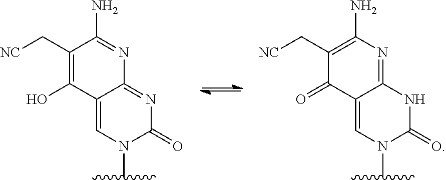 [SMILES: *n1cc2c(O)c(CC#N)c(N)nc2nc1=O ; *n1cc2c(=O)c(CC#N)c(N)nc-2[nH]c1=O]

In some examples, the photoredox reaction uses

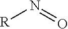

and light. The photoredox reaction may form an intermediate:

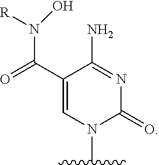

The intermediate may rearrange to form the product including:

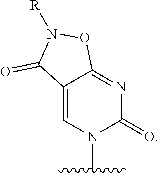

In some examples, the photoredox reaction uses

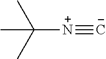

and light. The photoredox reaction may form an intermediate:

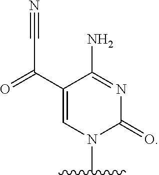 [SMILES: *n1cc(C(=O)C#N)c(N)nc1=O]

The intermediate may rearrange to form the product including:

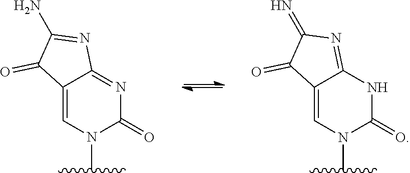 [SMILES: *n1cc2c(nc1=O)N=C(N)C2=O ; *N1C=C2C(=O)C(=N)N=C2NC1=O]

In some examples, the photoredox reaction further uses a photosensitizer.

In some examples, the photosensitizer is selected from the group consisting of: an iridium catalyst, a ruthenium complex, and an organic photosensitizer. In some examples, the iridium catalyst is selected from the group consisting of: fac-[Ir(ppy)₃], fac-[Ir(ppy)₃], and [Ir(dF(CF₃)ppy)₂(dtbbpy)]PF₆. In some examples, the ruthenium complex is selected from the group consisting of: Ru(bpz)₃(PF₆)₂, Ru(bpm)₃Cl₂, Ru(bpy)₃Cl₂, and Ru(phen)₃Cl₂. In some examples, the organic photosensitizer is selected from the group consisting of: Eosin Y, triphenylpyrylium tetrafluoroborate, and [Acr-Mes](ClO₄).

Some examples herein provide a method of modifying 5-methylcytosine (5-mC), 5-hydroxymethylcytosine (5-hmC), or 5-formylcytosine (5-fC) in a polynucleotide. The method may include oxidizing the 5-mC, 5-hmC, or 5-fC to 5-carboxylcytosine (5-caC); and using any of the above-provided methods to generate the product.

In some examples, a ten-eleven translocation (TET) dioxygenase is used to oxidize the 5-mC, 5-hmC, or 5-fC to 5-caC. In some examples, oxidizing the 5-mC, 5-hmC, or 5-fC to 5-carboxylcytosine (5-caC) includes contacting the 5-mC, 5-hmC, or 5-fC with one or more chemical reagents.

Some examples herein provide a method of detecting 5-methylcytosine (5-mC), 5-hydroxymethylcytosine (5-hmC), or 5-formylcytosine (5-fC) in a polynucleotide. The method may include modifying the 5-mC, 5-hmC, or 5-fC using the above-described method to generate a modified polynucleotide including the product; and detecting the 5-mC, 5-hmC, or 5-fC using the modified polynucleotide.

Some examples herein provide a method of modifying 5-methylcytosine (5-mC) in a polynucleotide. The method may include using a photoredox reaction to install a functional group at the 5-methyl group of the 5-mC. The installed functional group further may react with the 5-mC to form a product having at least two rings.

In some examples, the product has the structure:

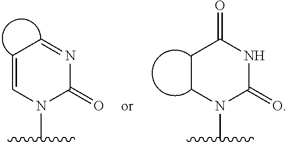 [SMILES: *n1cccnc1=O ; *N1CCC(=O)NC1=O]

In some examples, the photoredox reaction abstracts a hydrogen from the 5-methyl group, leaving behind a radical that is used to install the functional group.

In some examples, the photoredox reaction uses

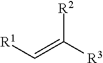

and light. In some examples, R¹ is an electron withdrawing group or includes an aryl group. In some examples, the electron withdrawing group is selected from the group consisting of aldehyde, ketone, ester, amide, hydrazido, cyano, and nitro. In some examples, R² and R³ promote formation of at least one of the two rings.

In some examples,

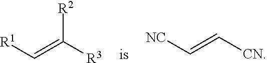

The photoredox reaction may form an intermediate:

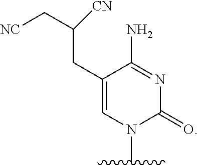 [SMILES: *n1cc(CC(C#N)CC#N)c(N)nc1=O]

The intermediate may rearrange to form the product including:

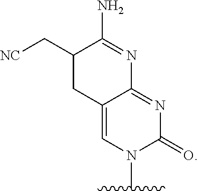 [SMILES: *n1cc2c(nc1=O)N=C(N)C(CC#N)C2]

In some examples, the photoredox reaction further uses N-hydroxy-phthalimide and a benzophenone derivative.

Some examples herein provide a method of detecting 5-methylcytosine (5-mC) in a polynucleotide. The method may include modifying the 5-mC using the method of any one of the preceding claims to generate a modified polynucleotide including the product; and detecting the 5-mC using the modified polynucleotide.

Some examples herein provide a method of modifying 5-methylcytosine (5-mC) or 5-hydroxymethylcytosine (5-hmC), in a polynucleotide. The method may include using a first photoredox reaction to oxidize the 5-mC or 5-hmC to 5-fC; and installing a functional group at the 5-position of the 5-fC. The installed functional group may further react with the 5-fC to form a product having at least two rings.

In some examples, a single reaction mixture is used to oxidize the 5-mC or 5-hmC to 5-fC and to install the functional group of the 5-fC. In some examples, Friedländer cyclization is used to install the functional group.

Some examples herein provide a polynucleotide including a modified 5-methylcytosine or derivative thereof having the structure

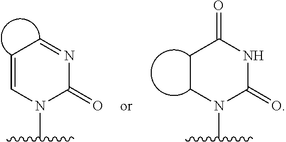 [SMILES: *n1cccnc1=O ; *N1CCC(=O)NC1=O]

Some examples herein provide a composition, including a polynucleotide in contact with

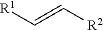

and light.

In some examples, R¹ includes an electron withdrawing group. In some examples, the electron withdrawing group is selected from the group consisting of aldehyde, ketone, ester, amide, hydrazido, cyano, and nitro.

In some examples, R¹ is an ester, and R² is H. In some examples, R¹ is an amide, and R² is ethanethiol. In some examples,

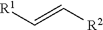

is diethyl ethylidenemalonate.

In some examples, the composition further includes a donor-acceptor complex. In some examples, the composition further includes a copper salt and an oxidant.

Some examples herein provide a composition, including a polynucleotide in contact with

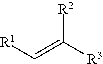

and light.

In some examples, R¹ is an electron withdrawing group or includes an aryl group. In some examples, the electron withdrawing group is selected from the group consisting of aldehyde, ketone, ester, amide, hydrazido, cyano, and nitro.

In some examples,

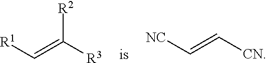

Some examples herein provide a composition, including a polynucleotide in contact with

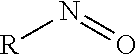

and light.

Some examples herein provide a composition, including a polynucleotide in contact with

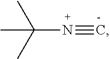

and light.

In some examples, any of the foregoing compositions may include a photosensitizer.

Some examples herein provide a method of selectively reducing the 5-6 double bond of 5-carboxylcytosine (5-caC), including using a photosensitizer and a reagent in a light-mediated reaction:

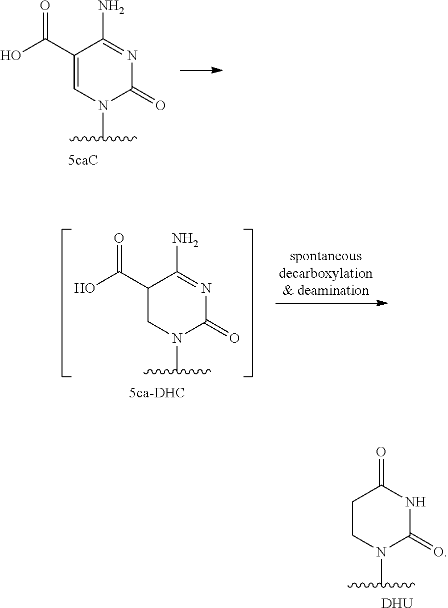

In some examples, the reagent includes a hydrogen donor or a reducing agent. In some examples, the hydrogen donor includes a reduced nicotinamide derivative or tris(trimethylsilyl silane) (TTMSS). In some examples, the reducing agent includes a Hantzsch ester. In some examples, the reducing agent includes flavin mononucleotide, riboflavin, lumiflavin, reduced nicotinamide adenine dinucleotide (NADH), reduced nicotinamide adenine dinucleotide phosphate (NADPH), 2′,3′,4′,5′-tetraacetylriboflavin, or tetrahydroxy diboron.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

### BRIEF DESCRIPTION OF DRAWINGS

FIG. 1 schematically illustrates an example flow of operations in a method for modifying methylcytosine or derivative thereof using a photoredox reaction.

FIG. 2 schematically illustrates example structures formed using operations described with reference to FIG. 1.

FIG. 3 schematically illustrates an example flow of operations in an alternative method for modifying methylcytosine using a photoredox reaction.

FIG. 4 schematically illustrates example structures formed using operations described with reference to FIG. 3.

FIG. 5 schematically illustrates an example flow of operations in an alternative method for modifying methylcytosine or hydroxymethylcytosine using a photoredox reaction.

FIG. 6 schematically illustrates example structures formed using operations described with reference to FIG. 5.

FIG. 7A schematically illustrates example hydrogen bonding between 5-carboxylcytosine and guanine in a double-stranded polynucleotide.

FIG. 7B schematically illustrates example hydrogen bonding between 5-carboxylcytosine, modified using a photoredox reaction in a manner such as provided herein, and adenine in a double-stranded polynucleotide.

FIG. 8 schematically illustrates example operations for detecting methylcytosine or derivative thereof in a polynucleotide using operations, structures, and hydrogen bonding such as described with reference to FIGS. 1, 2, and 7A-7B.

FIG. 9 schematically illustrates example operations for detecting methylcytosine in a polynucleotide using operations, structures, and hydrogen bonding such as described with reference to FIGS. 3 and 4.

FIG. 10 schematically illustrates example operations for detecting methylcytosine or hydroxymethylcytosine in a polynucleotide using operations, structures, and hydrogen bonding such as described with reference to FIGS. 5 and 6.

### DETAILED DESCRIPTION

Examples provided herein are related to methods of modifying methylcytosine or a derivative thereof using a photoredox reaction, and methods of using the same to detect the methylcytosine or derivative thereof in a polynucleotide.

In some examples provided herein, methylcytosine (mC), or a derivative thereof such as hydroxymethylcytosine (hmC) or formylcytosine (fC), in a polynucleotide may be detected using a workflow in which the mC, hmC, or fC is enzymatically or chemically oxidized to carboxylcytosine (caC), and a photoredox reaction is used to modify the caC to generate a product that includes two rings. The polynucleotide then may be amplified using polymerase chain reaction (PCR), during which the modified caC is amplified as thymine (T) and as such the mC, hmC, or fC is sequenced as T. In comparison, the unmethylated C is amplified, and sequenced, as C. Thus, any Cs in the sequence may be identified as corresponding to C because they had not been converted to T, while any mC, hmC, or fC in the sequence may be identified as corresponding to mC, hmC, or fC because they had been converted to T. In other examples provided herein, methylcytosine (mC) in a polynucleotide may be detected using a workflow in which a photoredox reaction is used to modify the mC to generate a product that includes two rings. The polynucleotide then may be amplified using polymerase chain reaction (PCR), during which the modified mC is amplified as thymine (T) and as such the mC is sequenced as T. In comparison, the unmethylated C is amplified, and sequenced, as C. Thus, any Cs in the sequence may be identified as corresponding to C because they had not been converted to T, while any mC in the sequence may be identified as corresponding to mC because they had been converted to T. Both of such schemes may be referred to as a “four-base” sequencing scheme because any unmethylated C is sequenced as C, providing the ability to obtain both sequence and methylation information from the processed polynucleotide. As provided herein, the present methods use reagents that are sufficiently water-soluble and mild as to be used with polynucleotides in a practical commercial implementation, and substantially without damaging the polynucleotides thus improving yield and accuracy of detecting mC, hmC, or fC while preserving the polynucleotide sequence itself as well.

First, some terms used herein will be briefly explained. Then, some example methods for modifying mC or its derivatives using a photoredox reaction, structures formed using such methods, and methods for detecting mC or its derivatives in a polynucleotide using the present subject matter, will be described.

### Terms

Unless defined otherwise, all technical and scientific terms used herein have the same meaning as is commonly understood by one of ordinary skill in the art. The use of the term “including” as well as other forms, such as “include,” “includes,” and “included,” is not limiting. The use of the term “having” as well as other forms, such as “have,” “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps, but may include additional steps. When used in the context of a compound, composition, or device, the term “comprising” means that the compound, composition, or device includes at least the recited features or components, but may also include additional features or components.

The terms “substantially,” “approximately,” and “about” used throughout this specification are used to describe and account for small fluctuations, such as due to variations in processing. For example, they may refer to less than or equal to ±10%, such as less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

As used herein, “hybridize” is intended to mean noncovalently associating a first polynucleotide to a second polynucleotide along the lengths of those polymers to form a double-stranded “duplex.” For instance, two DNA polynucleotide strands may associate through complementary base pairing. The strength of the association between the first and second polynucleotides increases with the complementarity between the sequences of nucleotides within those polynucleotides. The strength of hybridization between polynucleotides may be characterized by a temperature of melting (Tm) at which 50% of the duplexes disassociate from one another.

As used herein, the term “nucleotide” is intended to mean a molecule that includes a sugar and at least one phosphate group, and in some examples also includes a nucleobase. A nucleotide that lacks a nucleobase may be referred to as “abasic.” Nucleotides include deoxyribonucleotides, modified deoxyribonucleotides, ribonucleotides, modified ribonucleotides, peptide nucleotides, modified peptide nucleotides, modified phosphate sugar backbone nucleotides, and mixtures thereof. Examples of nucleotides include adenosine monophosphate (AMP), adenosine diphosphate (ADP), adenosine triphosphate (ATP), thymidine monophosphate (TMP), thymidine diphosphate (TDP), thymidine triphosphate (TTP), cytidine monophosphate (CMP), cytidine diphosphate (CDP), cytidine triphosphate (CTP), guanosine monophosphate (GMP), guanosine diphosphate (GDP), guanosine triphosphate (GTP), uridine monophosphate (UMP), uridine diphosphate (UDP), uridine triphosphate (UTP), deoxyadenosine monophosphate (dAMP), deoxyadenosine diphosphate (dADP), deoxyadenosine triphosphate (dATP), deoxythymidine monophosphate (dTMP), deoxythymidine diphosphate (dTDP), deoxythymidine triphosphate (dTTP), deoxycytidine diphosphate (dCDP), deoxycytidine triphosphate (dCTP), deoxyguanosine monophosphate (dGMP), deoxyguanosine diphosphate (dGDP), deoxyguanosine triphosphate (dGTP), deoxyuridine monophosphate (dUMP), deoxyuridine diphosphate (dUDP), and deoxyuridine triphosphate (dUTP).

As used herein, the term “nucleotide” also is intended to encompass any nucleotide analogue which is a type of nucleotide that includes a modified nucleobase, sugar and/or phosphate moiety compared to naturally occurring nucleotides. Example modified nucleobases include xanthine, hypoxanthine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thiouracil, 2-thiothymine, 2-thiocytosine, 5-halouracil, 5-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. As is known in the art, certain nucleotide analogues cannot become incorporated into a polynucleotide, for example, nucleotide analogues such as adenosine 5′-phosphosulfate. Nucleotides may include any suitable number of phosphates, e.g., three, four, five, six, or more than six phosphates.

As used herein, the term “polynucleotide” refers to a molecule that includes a sequence of nucleotides that are bonded to one another. A polynucleotide is one nonlimiting example of a polymer. Examples of polynucleotides include deoxyribonucleic acid (DNA), ribonucleic acid (RNA), and analogues thereof. A polynucleotide may be a single stranded sequence of nucleotides, such as RNA or single stranded DNA, a double stranded sequence of nucleotides, such as double stranded DNA, or may include a mixture of a single stranded and double stranded sequences of nucleotides. Double stranded DNA (dsDNA) includes genomic DNA, and PCR and amplification products. Single stranded DNA (ssDNA) can be converted to dsDNA and vice-versa. Polynucleotides may include non-naturally occurring DNA, such as enantiomeric DNA. The precise sequence of nucleotides in a polynucleotide may be known or unknown. The following are examples of polynucleotides: a gene or gene fragment (for example, a probe, primer, expressed sequence tag (EST) or serial analysis of gene expression (SAGE) tag), genomic DNA, genomic DNA fragment, exon, intron, messenger RNA (mRNA), transfer RNA, ribosomal RNA, ribozyme, cDNA, recombinant polynucleotide, synthetic polynucleotide, branched polynucleotide, plasmid, vector, isolated DNA of any sequence, isolated RNA of any sequence, nucleic acid probe, primer or amplified copy of any of the foregoing.

The terms “polynucleotide” and “oligonucleotide” are used interchangeably herein. The different terms are not intended to denote any particular difference in size, sequence, or other property unless specifically indicated otherwise. For clarity of description the terms may be used to distinguish one species of polynucleotide from another when describing a particular method or composition that includes several polynucleotide species.

As used herein, the term “methylcytosine” or “mC” refers to cytosine that includes a methyl group (—CH₃ or -Me). The methyl group may be located at the 5 position of the cytosine, in which case the mC may be referred to as 5-mC.

As used herein, unless context makes clear otherwise, the term “C*” generally refers to a cytosine the 5 prime position of which is coupled to a functional group X where the functional group is capable of reacting with the cytosine to form a product having two rings.

As used herein, unless context makes clear otherwise, the term “T*” generally refers a bicyclic product having a hydrogen bonding pattern that is sufficiently similar to T as to pair with A, formed from cytosine reacting with functional group X.

As used herein, a “derivative” of methylcytosine refers to methylcytosine having an oxidized methyl group. A nonlimiting example of an oxidized methyl group is hydroxymethyl (—CH₂OH), in which case the mC derivative may be referred to as hydroxymethylcytosine or hmC. Another nonlimiting example of an oxidized methyl group is formyl group (—CHO) in which case the mC derivative may be referred to as formylcytosine or fC. Another nonlimiting example of an oxidized methyl group is carboxyl (—COOH), in which case the mC derivative may be referred to as carboxylcytosine or caC. The oxidized methyl group may be located at the 5 position of the cytosine, in which case the hmC may be referred to as 5-hmC, the fC may be referred to as 5-fC, or the caC may be referred to as 5-caC. The fC optionally may be present in an acetal form (—CH(OH)₂). The caC optionally may be present in a salt form (—COO⁻).

As used herein, the terms “electron donating group,” “electron-donor,” and the like are intended to refer to a group that releases electron density from itself to adjacent atoms, thereby increasing the electron density of the adjacent atoms.

As used herein, the terms “electron withdrawing group,” “electron-acceptor,” and the like are intended to refer to a group that draws electron density from adjacent atoms to itself, thereby reducing electron density of the adjacent atoms.

As used herein, the term “aqueous solution” is intended to refer to any solution in which water functions as a solvent.

As used therein, the term “photoredox reaction” is intended to refer to a reaction that transfers at least one electron from a first atom to a second atom responsive to exposure to light. The first atom may be part of a first molecule, and the second atom may be part of a second molecule that is different from the first molecule. Alternatively, the first atom and the second atom may be part of the same molecule as one another.

### Methods of Modifying Methylcytosine or a Derivative Thereof Using a Photoredox Reaction

Some examples provided herein relate to modifying methylcytosine (5-mC) or a derivative thereof (e.g., 5-hmC or 5-fC) using a photoredox reaction.

More specifically, the present inventors have recognized that in some examples, 5-mC, 5-hmC, or 5-fC in a polynucleotide may be converted to caC, and the caC selectively subjected to a photoredox reaction to form a product. In other examples, such as described further below, the 5-mC may be subjected to a photoredox reaction to form a product.

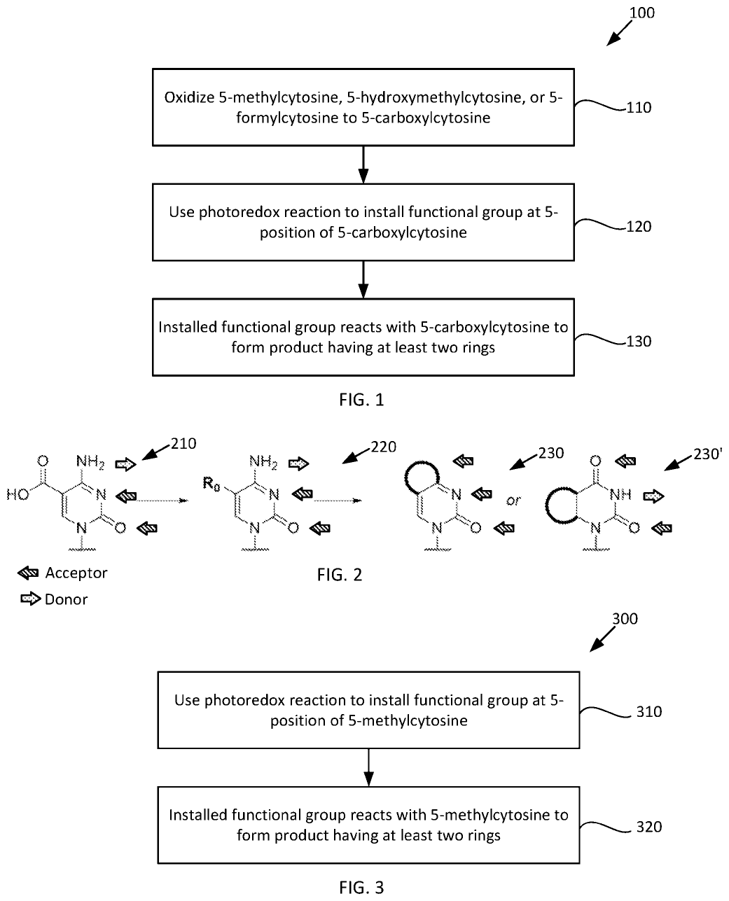

FIG. 1 schematically illustrates an example flow of operations in a method for modifying methylcytosine or derivative thereof using a photoredox reaction, and FIG. 2 schematically illustrates example structures formed using operations described with reference to FIG. 1. Referring first to FIG. 1, method 100 optionally may include oxidizing 5-mC, 5-hmC, or 5-fC to 5-caC (operation 110). The oxidation may be performed using any suitable combination of chemical and/or enzymatic reagents. In some examples using an enzymatic reagent, a ten-eleven translocation (TET) dioxygenase is used to oxidize the 5-mC, 5-hmC, or 5-fC to 5-caC. In some nonlimiting examples using one or more chemical reagents, 5-mC may be oxidized to 5-caC using menadione, ultraviolet (UV) radiation at 365 nm, under oxygen, followed by 2,2,6,6-tetramethyl-1-piperidinyloxy free radical (TEMPO)/bis(acetoxyiodobenzene) (BAIB) in a manner such as described in Kore et al., “Concise synthesis of 5-methyl, 5-formyl, and 5-carboxy analogues of 2′-deoxycytidine-5′-triphosphate,” Tetrahedron letters 54 (39): 5325-5327 (2013), the entire contents of which are incorporated by reference herein. In other nonlimiting examples using one or more chemical reagents, 5-hmC or 5-fC may be oxidized to 5-caC using TEMPO/BAIB in a manner such as described in Sun et al., “Efficient synthesis of 5-hydroxymethyl-, 5-formyl-, and 5-carboxyl-2′-deoxycytidine and their triphosphates,” RSC Advances 4 (68): 36036-36039 (2014), the entire contents of which are incorporated by reference herein. In still other nonlimiting examples using one or more chemical reagents, an iron (IV)-oxo complex is used to oxidize 5-mC to 5-caC in a manner such as described in Schmidl et al., “Biomimetic iron complex achieves TET enzyme reactivity,” Angewandte Chemie Int'l Ed. 60 (39): 21457-21463 (2021), the entire contents of which are incorporated by reference herein.

The 5-mC, 5-hmC, or 5-fC may be present in a polynucleotide, and the polynucleotide may be present in an extracellular sample. As such, the 5-caC resulting from operation 110 may be present in the polynucleotide. An example structure of caC, which may be present in a polynucleotide and resulting from operation 110, is illustrated at operation 210 in FIG. 2.

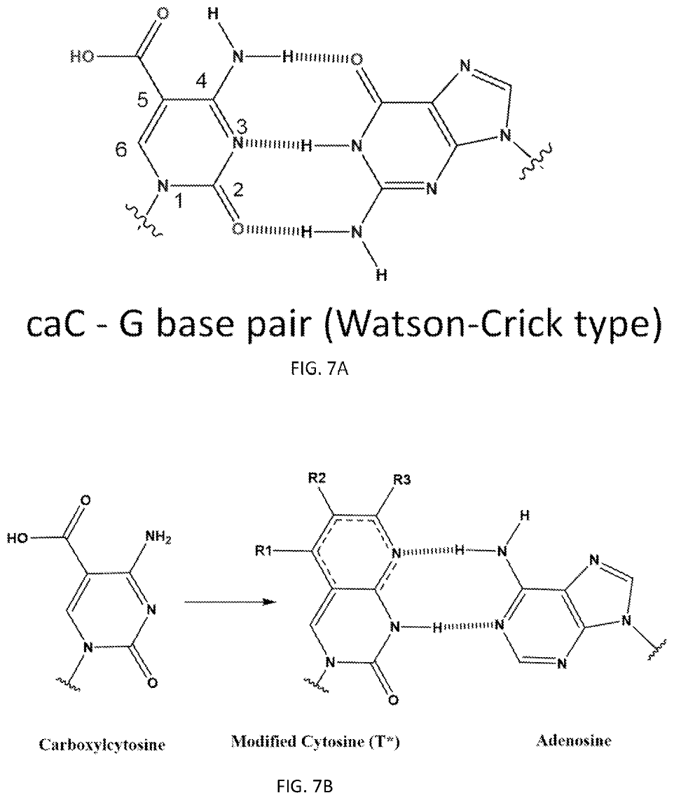

Referring again to FIG. 1, method 100 may include using a photoredox reaction to install a functional group at the 5-position of the 5-caC (operation 120). In some examples, the installed functional group replaces the 5-carboxyl group to provide functional group R₀ illustrated at operation 220 of FIG. 2, while in other examples, the installed functional group modifies the 5-carboxyl group to provide functional group R₀ illustrated at operation 220 of FIG. 2. The photoredox reaction may be performed using any suitable combination of chemical reagent(s) and wavelength(s) of light. Nonlimiting examples of redox reactions and functional groups are provided further below. Method 100 also may include the installed functional group reacting with the 5-caC to form a product having at least two rings (operation 130). In some examples, each of the rings may be either a 5-membered ring or a 6-membered ring. Example structure of products resulting from operation 130, which products may be present in a polynucleotide, respectively are illustrated at operations 230 and 230′ in FIG. 2. Note that the product illustrated at 230 in FIG. 2 may be expected to have a hydrogen bonding pattern that is sufficiently similar to T as to pair with A and thus be read out as a T, e.g., in a manner similar to that described in Zhu et al., “Single-cell 5-formylcytosine landscapes of mammalian early embryos and ESCs at single-base resolution,” Cell Stem Cell 20: 720-731 (2017), or in Xia et al., “Bisulfite-free, base-resolution analysis of 5-formylcytosine at the genome scale,” Nature Methods 12: 1047-1050 (2015), the entire contents of both of which are incorporated by reference herein. The product illustrated at 230′ may be expected to have a hydrogen bonding pattern that is sufficiently similar to T as to pair with A and thus be read out as a T, e.g., in a manner such as described with reference to FIG. 7B.

The reaction of operation 130 may be performed subsequently to operation 120, or may be performed concurrently with operation 120. In some examples, operation 130 includes rearrangement of the intermediate generated using operation 120. In some examples, the product generated using operations 120 and 130, and illustrated at 230 in FIG. 2, may be able to tautomerize between the following two structures:

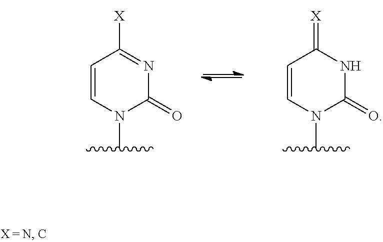

Note that the tautomer illustrated on the right side of the scheme immediately above may be expected to have a similar pattern of hydrogen bonding as described in greater detail below with reference to FIG. 7B. In other examples, the product generated using operations 120 and 130, and illustrated at 230 in FIG. 2 may have the structure:

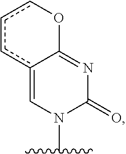

which may be expected to have a pattern of hydrogen bonding similar to that described in Zhu et al., “Single-cell 5-formylcytosine landscapes of mammalian early embryos and ESCs at single-base resolution,” Cell Stem Cell 20: 720-731 (2017), or in Xia et al., “Bisulfite-free, base-resolution analysis of 5-formylcytosine at the genome scale,” Nature Methods 12: 1047-1050 (2015).

Additional example structures that may be formed using operations described with reference to FIG. 1 may be envisioned. For example, the following structure corresponds to the product of operation 230 including two rings, in which each of the rings includes a six-membered ring:

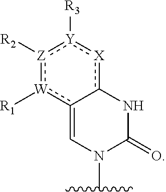

As another example, the following structure corresponds to the product of operation 230 including two rings, in which one of the rings includes a five membered ring and the other includes a six-membered ring:

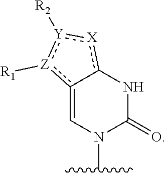

In the two structures illustrated immediately above, X can be N or C; W, Y, and Z independently can be C, N, O, S, or Se; and R₁, R₂, and R₃ independently can be H or a substituent. The two structures immediately above may be expected to have patterns of hydrogen bonding similar to those described in Zhu et al., “Single-cell 5-formylcytosine landscapes of mammalian early embryos and ESCs at single-base resolution,” Cell Stem Cell 20: 720-731 (2017), or in Xia et al., “Bisulfite-free, base-resolution analysis of 5-formylcytosine at the genome scale,” Nature Methods 12: 1047-1050 (2015).

As still another example, the following structure corresponds to the product of operation 230′ including two rings, in which each of the rings includes a six-membered ring:

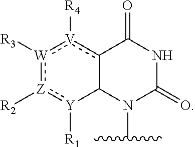

As yet another example, the following structure corresponds to the product of operation 230′ including two rings, in which one of the rings includes a five membered ring and the other includes a six-membered ring:

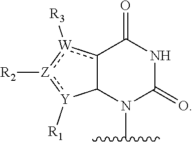

In the two structures illustrated immediately above, V, W, Y, and Z independently can be C, N, O, S, or Se; and R₁, R₂, R₃, and R₄ independently can be H or a substituent. The two structures immediately above may be expected to have patterns of hydrogen bonding similar to those described with reference to FIG. 7B.

Some nonlimiting examples of photoredox reactions that may be used to perform operation 120 using 5-caC, and result in the product of operation 130, now will be described. Further below, other types of photoredox reactions will be described that may be used to perform other operations on methylcytosine or other methylcytosine derivatives.

In some examples, the photoredox reaction of operation 120 includes a photoinduced decarboxylative radical reaction replacing the 5-carboxyl group with a functional group R₀:

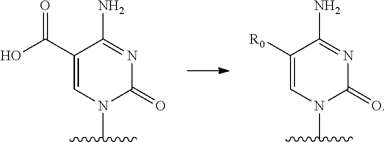

The functional group R₀ is selected such that, when installed in the 5-caC to form an intermediate (which intermediate may be referred to as 5-RC), the functional group may react with one or more other portions of the intermediate to form a product having two rings, such as:

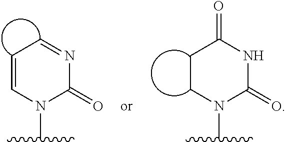 [SMILES: *n1cccnc1=O ; *N1CCC(=O)NC1=O]

As noted above with reference to FIGS. 1 and 2, the functional group may form a ring after being installed, or may form a ring while being installed.

Any suitable reagent(s) may be used, in addition to light, to perform the photoinduced decarboxylative radical reaction to install the functional group, such as

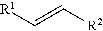

and a photocatalyst and/or photosensitizer. R¹ may include an electron withdrawing group, such as aldehyde, ketone, ester, amide, hydrazido, cyano, or nitro. It can be useful for R¹ to be an electron withdrawing group (EWG) so that the alkene group is polarized and electron-deficient in order to be able to react with the aryl radical generated by the photoredox reaction. The EWG can also promote the further rearrangement reaction. R² may promote formation of at least one of the two rings. For example, R² may be CH₂—XH, CH₂—CH₂—XH, where X is S, Se, N, or O. Alternatively, R² may be a cyano group or another electron withdrawing group such as described for R¹. Nonlimiting examples of photocatalysts and/or photosensitizers that may be used include: phenanthrene in combination with 1,4-dicyanobenzene, biphenyl in combination with 1,4-dicyanonaphthalene, and biphenyl in combination with 9,10-dicyanoanthracene. The wavelength of light suitably may be selected to initiate the photoinduced decarboxylative radical reaction. In some examples, the light may be in the visible portion of the spectrum, e.g., may be in the range of about 380 nm to about 700 nm, e.g., may be in the range of about 380 nm to about 500 nm, e.g., may be in the range of about 380 nm to about 420 nm, or may be in the range of about 450 nm to about 700 nm. In other examples, the light may be in the ultraviolet portion of the spectrum, e.g., may be in the range of about 200 nm to about 380 nm, e.g., may be in the range of about 250 nm to about 350 nm. Some nonlimiting examples of photoinduced decarboxylative radical reactions, and reagents for use in such reactions, now will be provided.

In some examples, R¹ is an ester, and R² is H. For example,

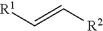

may be

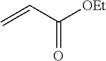

which, when reacted with 5-caC in a photoredox reaction in the presence of a suitable photocatalyst and/or photosensitizer (e.g., biphenyl and 1,4-dicyanonapthalene), NaOH, and blue light, forms an intermediate:

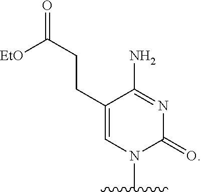 [SMILES: *n1cc(CCC(=O)OCC)c(N)nc1=O]

The intermediate may rearrange to form the bicyclic product including:

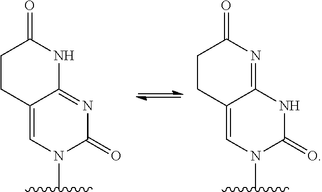 [SMILES: *n1cc2c(nc1=O)NC(=O)CC2 ; *N1C=C2CCC(=O)N=C2NC1=O]

The cyclic product of such rearrangement may be present in two tautomeric forms at equilibrium, e.g., between forms such as illustrated above where a hydrogen is located on the N4 or the N3 of the pyrimidine ring. At least the tautomer on the right may suitably hydrogen bond to A, and thus may be read out as T.

In certain other examples, R¹ is an amide, and R² is ethanethiol. Illustratively,

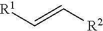

may be

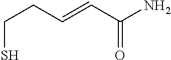

which, when reacted with 5-caC in a photoredox reaction, forms an intermediate:

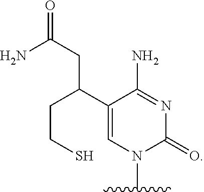 [SMILES: *n1cc(C(CCS)CC(N)=O)c(N)nc1=O]

The intermediate may rearrange to form the product including:

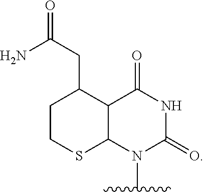 [SMILES: *N1C(=O)NC(=O)C2C(CC(N)=O)CCSC21]

Example conditions and reagents for this scheme are shown below:

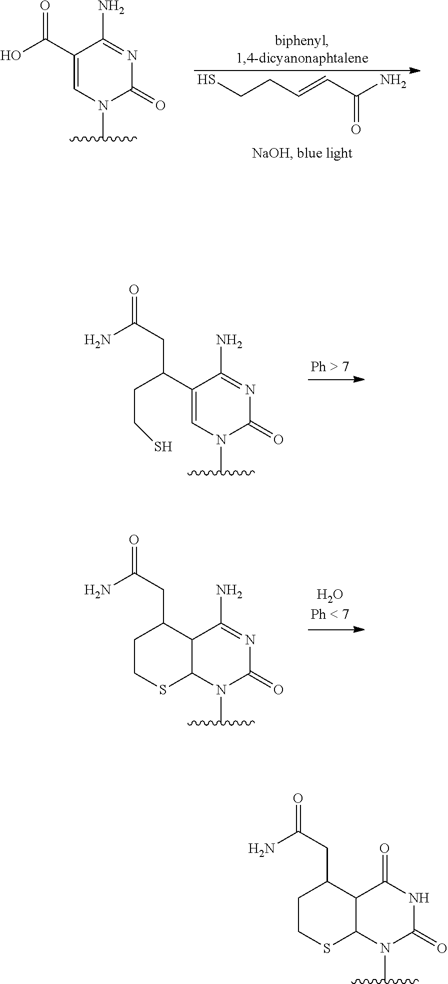 [SMILES: *n1cc(C(=O)O)c(N)nc1=O ; NC(=O)/C=C/CCS ; *n1cc(C(CCS)CC(N)=O)c(N)nc1=O ; *N1C(=O)N=C(N)C2C(CC(N)=O)CCSC21 ; *N1C(=O)NC(=O)C2C(CC(N)=O)CCSC21]

Note that the product of the present example may not tautomerize. Instead, the nucleophilic addition on the 6 position of the pyrimidine ring will trigger a deamination reaction on the position 4 of the pyrimidine ring.

In certain other examples,

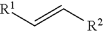

may be diethyl ethylidenemalonate, which when reacted with 5-caC in a photoredox reaction, forms an intermediate:

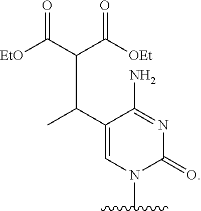 [SMILES: *n1cc(C(C)C(C(=O)OCC)C(=O)OCC)c(N)nc1=O]

The intermediate may rearrange to form the product including:

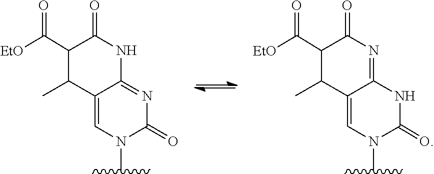 [SMILES: *n1cc2c(nc1=O)NC(=O)C(C(=O)OCC)C2C ; *N1C=C2C(=NC(=O)C(C(=O)OCC)C2C)NC1=O]

In this example, the photoredox reaction may be conducted using the photocatalysts Cu(MeCN)₄BF₄ or Cu(OTf)₂ with 1-fluoro-2,4,6-trimethylpyridinium tetrafluoroborate and irradiation at 365 nm. The cyclic product of such rearrangement may be present in two tautomeric forms at equilibrium, e.g., between forms such as illustrated above where a hydrogen is located on the N4 or the N3 of the pyrimidine ring. At least the tautomer on the right may suitably hydrogen bond to A, and thus may be read out as T.

While non-limiting examples of photocatalysts, photosensitizers, and other reagents and conditions are provided above, it will be appreciated that in photoinduced decarboxylative radical reactions such as described herein, any suitable reagent(s) or condition(s) may be used to facilitate such reactions. For example, the reactions may use any suitable photocatalyst(s) and/or photosensitizer(s), such as any suitable metal complexes, organophotocatalysts, donor-acceptor complexes, nanoparticles/polyoxometallates, or the like. For example, the photoinduced decarboxylative radical reaction optionally may use a donor-acceptor complex. Such a complex may be used to generate a radical cation that facilitates photoinduced decarboxylation at relatively mild temperature (e.g., about 20-40° C.), optionally under basic conditions. Any suitable combination of donor(s) and acceptor(s) may be used. Illustratively, the donor may be selected from the group consisting of: biphenyl and phenanthrene. Illustratively, the acceptor may be selected from the group consisting of: 1,4-dicyanonaphthalene, 9,10-dicyanoanthracene, and 1,4-dicyanobenzene. For further details regarding use of a donor-acceptor complex to facilitate photoinduced decarboxylative radical reactions, see Kubosaki et al., “Visible- and UV-light-induced decarboxylative radical reactions of benzoic acids using organic photoredox catalysts,” Journal of Organic Chemistry 85: 5362-5369 (2020), the entire contents of which are incorporated by reference herein.

As another example, the present photoinduced decarboxylative radical reaction may use a copper salt and an oxidant. The copper salt may form a light-excitable complex with the carboxylate, which upon irradiation may dissociate to form a decarboxylated radical and CO₂, and reduce the copper salt. The oxidant may be used to re-oxidize the copper salt for use in another such reaction. Any suitable copper salts may be used, such as a Cu(I) salt or a Cu(II) salt. Nonlimiting examples of Cu(I) salts include those selected from the group consisting of: CuBr, Cu(OAc), and Cu(OTf). Nonlimiting examples of Cu(II) salts include those selected from the group consisting of: CuBr₂, Cu(OAc)₂, and Cu(OTf)₂. Nonlimiting examples of oxidants include those selected from the group consisting of N-fluorobenzene sulfonamide, Selectfluor, 1-fluoropyridinium or derivative thereof (illustratively, 1-fluoro-2,4,6-trimethylpyridinium tetrafluoroborate), and dicumyl peroxide. For further details regarding use of a copper salt and oxidant to facilitate photoinduced decarboxylative radical reactions, see Chen et al., “Ligand-to-copper charge transfer: A general catalytic approach to aromatic decarboxylative functionalization,” doi: 10.26434/chemrxiv.14451117.v1, 15 pages (2021), the entire contents of which are incorporated by reference herein.

It will be appreciated that photoredox reactions other than photoinduced decarboxylative radical reactions suitably may be used to install a functional group to 5-caC that may be used to form a product having at least two rings in a manner such as described with reference to FIGS. 1 and 2. For example, the photoredox reaction instead may include a photoinduced deoxygenative radical reaction that functionalizes the 5-carboxyl group to include a functionalized 5-acyl group 5-acyl group-(CO)R₀:

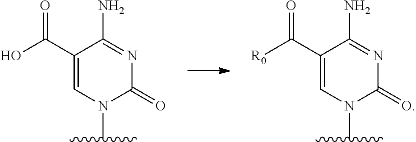

The functional group R₀ is selected such that, when installed in the 5-caC to form an intermediate (which intermediate may be referred to as 5-(CO)RC), the functional group may react with one or more other portions of the intermediate to form a product having two rings, such as:

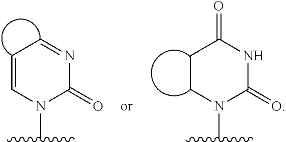 [SMILES: *n1cccnc1=O ; *N1CCC(=O)NC1=O]

As noted above with reference to FIGS. 1 and 2, the functional group may form a ring after being installed, or may form a ring while being installed.

Any suitable reagent(s) may be used, in addition to light, to perform the photoinduced deoxygenative radical reaction to install the functional group, such as

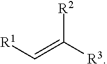

R¹ optionally may include an electron withdrawing group, such as aldehyde, ketone, ester, amide, hydrazido, cyano, or nitro. In some examples, R¹ may be or include an aryl ring, so an EWG may not be required for the initial radical reaction, although an EWG may make coupling with the acyl radical more favorable. Additionally, having an EWG or electrophilic group may facilitate subsequent rearrangements that eventually include a hydrogen bonding pattern that hydrogen bonds to A and thus may be read as T. R² and R³ may promote formation of at least one of the two rings. The wavelength of light suitably may be selected to initiate the photoinduced deoxygenative radical reaction. In some examples, the light may be in the visible portion of the spectrum, e.g., may be in the range of about 380 nm to about 700 nm, e.g., may be in the range of about 380 nm to about 500 nm, e.g., may be in the range of about 380 nm to about 420 nm, or in the range of about 450 nm to about 700 nm. In other examples, the light may be in the ultraviolet portion of the spectrum, e.g., may be in the range of about 200 nm to about 380 nm, e.g., may be in the range of about 250 nm to about 350 nm. Some nonlimiting examples of photoinduced deoxygenative radical reactions, and reagents for use in such reactions, now will be provided.

In some examples,

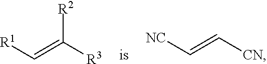

which when reacted with 5-caC in a photoredox reaction in the presence of a suitable photocatalyst and blue light, forms an intermediate:

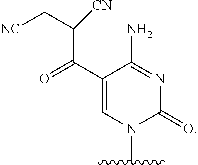 [SMILES: *n1cc(C(=O)C(C#N)CC#N)c(N)nc1=O]

The intermediate may rearrange to form the product including:

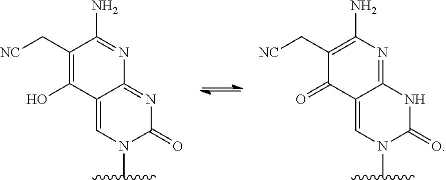 [SMILES: *n1cc2c(O)c(CC#N)c(N)nc2nc1=O ; *n1cc2c(=O)c(CC#N)c(N)nc-2[nH]c1=O]

Note that the product of the present example may be present in two tautomeric forms at equilibrium, e.g., between forms such as illustrated above where a hydrogen is located on the N3 of the pyrimidine ring or located on the oxygen on the second ring. At least the tautomer on the right (and potentially also the tautomer on the left) may suitably hydrogen bond to A, and thus may be read out as T.

Still other examples need not necessarily use

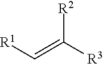

as a reactant. Illustratively, the photoredox reaction may use

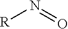

which, when reacted with the 5-caC in the presence of a suitable photocatalyst and blue light in the photoredox reaction, forms an intermediate:

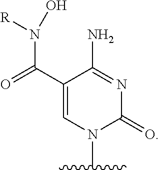

The intermediate may rearrange to form the product including:

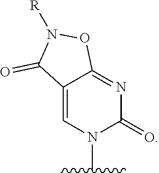

Note that the product of the present example may not tautomerize in a manner similar to that described with reference to other examples herein.

In yet another example, the photoredox reaction uses

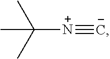

which, when reacted with the 5-caC in the photoredox reaction in the presence of a photocatalyst and blue light, forms an intermediate:

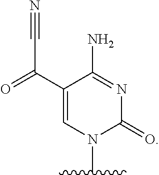 [SMILES: *n1cc(C(=O)C#N)c(N)nc1=O]

The intermediate may rearrange to form the product including:

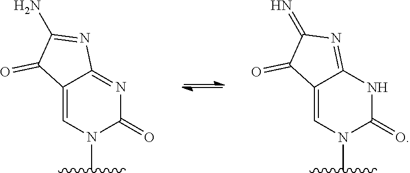 [SMILES: *n1cc2c(nc1=O)N=C(N)C2=O ; *N1C=C2C(=O)C(=N)N=C2NC1=O]

Note that the product of the present example may be present in two tautomeric forms at equilibrium, e.g., between forms such as illustrated above where a hydrogen is located on the N3 of the pyrimidine ring or on the exocyclic amino group. At least the tautomer on the right (and potentially also the tautomer on the left) may suitably hydrogen bond to A, and thus may be read out as T.

It will be appreciated that in photoinduced deoxygenative radical reactions such as described herein, any suitable additional reagent(s) or condition(s) may be used to facilitate such reactions. For example, the reactions may use a photosensitizer. The photosensitizer may be selected from the group consisting of: an iridium catalyst, a ruthenium complex, and an organic photosensitizer. Illustratively, the iridium catalyst is selected from the group consisting of: fac-[Ir(ppy)₃], fac-[Ir(ppy)₃], and [Ir(dF(CF₃)ppy)₂(dtbbpy)]PF₆. In some examples, the ruthenium complex is selected from the group consisting of: Ru(bpz)₃(PF₆)₂, Ru(bpm)₃Cl₂, Ru(bpy)₃Cl₂, and Ru(phen)₃Cl₂. In some examples, the organic photosensitizer is selected from the group consisting of: Eosin Y, triphenylpyrylium tetrafluoroborate, and [Acr-Mes](ClO₄). Nonlimiting examples of reagents and conditions for use in photoinduced deoxygenative radical reactions may be found in the following references, the entire contents of each of which are incorporated by reference herein: Bergonzini et al., “Acyl radicals from aromatic carboxylic acids by means of visible-light photoredox catalysis,” Angewandte Communications Int'l Ed. 54: 14066-14069 (2015); Zhang et al., “Photoredox-catalyzed hydroacylation of olefins employing carboxylic acids and hydrosilanes,” Organic Letters 19: 3430-3433 (2017); and Zhang et al., “A general deoxygenation approach for synthesis of ketones from aromatic carboxylic acids and alkenes,” Nature Communications 9: 3517 (2018). It will be appreciated that any of the present photoredox reactions may use such a photosensitizer, and that the photosensitizer is not limited to use in photoinduced deoxygenative radical reactions.

It will be appreciated that 5-caC may be modified in still other ways using a photoredox reaction. For example, the 5-6 double bond of 5-caC may be selectively reduced through use of a photosensitizer and suitable reagent in a light-mediated reaction such as illustrated below:

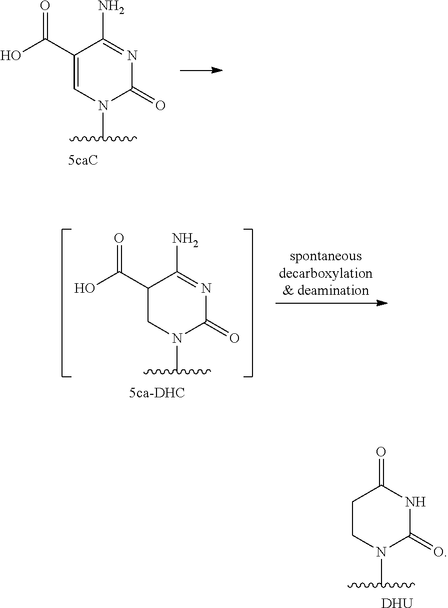

Example reagents for such a scheme include an H donor (such as a reduced nicotinamide derivative, tris(trimethylsilyl silane) (TTMSS), or a reducing agent, such as a Hantzsch ester). Other nonlimiting examples of reducing agents that may be used in photoredox catalysis include reducing agents such as: flavin mononucleotide, riboflavin, lumiflavin, reduced nicotinamide adenine dinucleotide (NADH), reduced nicotinamide adenine dinucleotide phosphate (NADPH), 2′,3′,4′,5′-tetraacetylriboflavin, and tetrahydroxydiboron. Such reducing agents may be expected to be less toxic than nucleophiles such as thiols. Additionally, thiols are prone to oxidation and dimerization and so may be insufficiently stable in solution, and may interfere with enzymes. In one nonlimiting example, the 5-6 double bond of 5-caC may be selectively reduced using [Ru(bpy)₃]Cl, 1-benzyl-1,4-dihydronicotinamide, and visible light irradiation (>470 nm) in a manner such as described in Pac et al., “Ru(bpy)₃²⁺-mediated photoreduction of olefins with 1-benzyl-1,4-dihydronicotinamide: A mechanistic probe for electron-transfer reactions of NAD(P)H-model compounds,” J. Am. Chem. Soc. 103: 6495-6497 (1981), the entire contents of which are incorporated by reference herein. In another nonlimiting example, the 5-6 double bond of 5-caC may be selectively reduced using [Ir(ppy)₂(dtbbpy)]PF₆, Hantzsch ester, and visible light irradiation (blue LED) in a manner such as described in Larionova et al., “Efficient reduction of electron-deficient alkenes enabled by a photoinduced hydrogen atom transfer,” doi: 10.26434/chemrxiv.12380330.v2, 5 pages (2020), the entire contents of which are incorporated by reference herein.

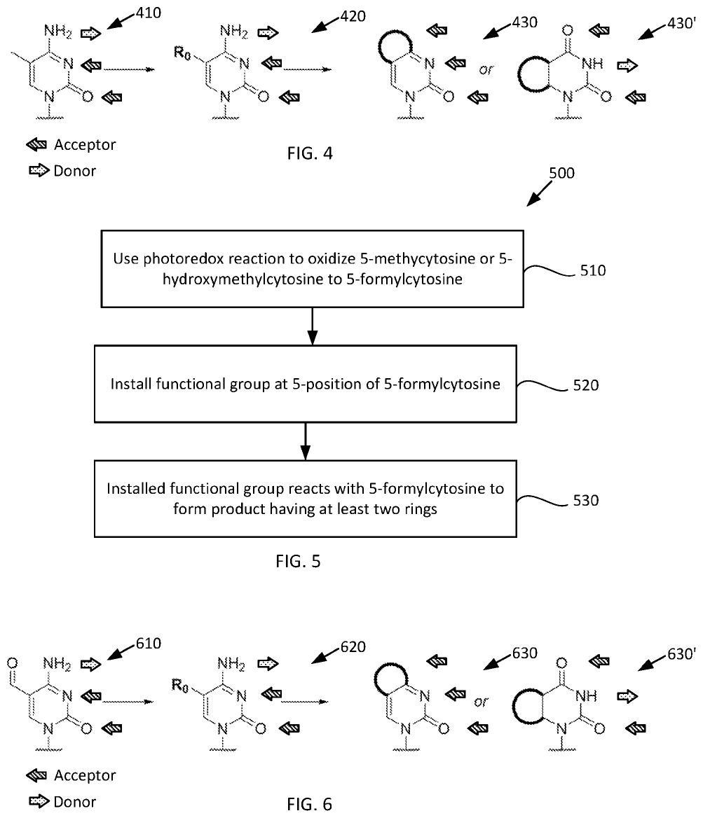

As noted further above, in other examples, 5-mC may be modified using a photoredox reaction. FIG. 3 schematically illustrates an example flow of operations in an alternative method for modifying methylcytosine using a photoredox reaction, and FIG. 4 schematically illustrates example structures formed using operations described with reference to FIG. 3. Method 300 illustrated in FIG. 3 may include using a photoredox reaction to install a functional group at the 5-methyl group of the 5-mC (operation 310). The 5-mC such as illustrated at operation 410 of FIG. 4 may be present in a polynucleotide, and the polynucleotide may be present in an extracellular sample. In some examples, the installed functional group modifies the 5-methyl group to provide functional group R₀ illustrated at operation 420 of FIG. 4. The photoredox reaction may be performed using any suitable combination of chemical reagent(s) and wavelength(s) of light. Nonlimiting examples of redox reactions and functional groups are provided further below. Method 300 also may include the installed functional group further reacting with the 5-mC to form a product having at least two rings (operation 320). Example structure of products resulting from operation 320, which products may be present in a polynucleotide, respectively are illustrated at operations 430 and 430′ in FIG. 4, and are shown further below as well:

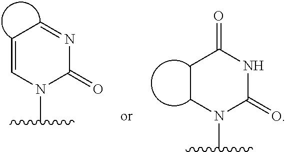 [SMILES: *n1cccnc1=O ; *N1CCC(=O)NC1=O]

The reaction of operation 320 may be performed subsequently to operation 310, or may be performed concurrently with operation 310. In some examples, operation 320 includes rearrangement of the intermediate generated using operation 310.

Some nonlimiting examples of photoredox reactions that may be used to perform operation 310 using 5-mC, and result in the product of operation 320, now will be described.

In some examples, the photoredox reaction abstracts a hydrogen from the 5-methyl group, leaving behind a radical that is used to install the functional group. Any suitable combination of reagent(s) and light may be used to perform such H-abstraction. Illustratively, the photoredox reaction may use

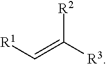

R¹ may be an electron withdrawing group, e.g., may be selected from the group consisting of aldehyde, ketone, ester, amide, hydrazido, cyano, and nitro. In other examples, R¹ may be or include an aryl ring, so an EWG may not be required for the initial radical reaction, although an EWG may make coupling with the acyl radical more favorable. Additionally, having an EWG or electrophilic group may facilitate subsequent rearrangements that eventually include a hydrogen bonding pattern that hydrogen bonds to A and thus may be read as T. R² and R³ may promote formation of at least one of the two rings, in a manner similar as described for the 5-caC photoredox reactions.

In yet another example,

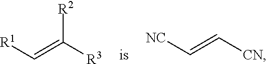

which when reacted with 5-mC in the presence of a photocatalyst and blue light in the photoredox reaction forms an intermediate:

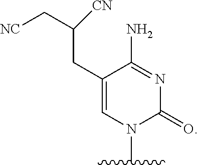 [SMILES: *n1cc(CC(C#N)CC#N)c(N)nc1=O]

The intermediate rearranges to form the product including

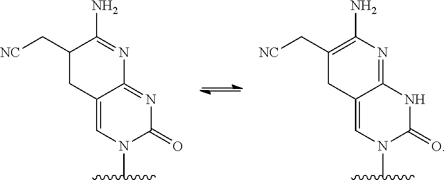 [SMILES: *n1cc2c(nc1=O)N=C(N)C(CC#N)C2 ; *N1C=C2CC(CC#N)=C(N)N=C2NC1=O]

Note that the product of the present example may be present in two tautomeric forms at equilibrium, e.g., between forms such as illustrated above where a hydrogen is located on the N3 of the pyrimidine ring or located on the second ring. At least the tautomer on the right (and potentially also the tautomer on the left) may suitably hydrogen bond to A, and thus may be read out as T.

Other nonlimiting examples for

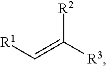

and reaction products that may be formed using such reagent, are provided above with reference to the 5-caC examples.

Additionally, similarly as described for the 5-caC examples, any suitable reagent(s) may be used to facilitate the photoredox reaction. Illustratively, the photoredox reaction optionally further may use N-hydroxy-phthalimide and a benzophenone derivative. The benzophenone derivative may be used for a proton-coupled electron transfer from N-hydroxy-phthalimide to generate a phthalimide-N-oxyl radical that facilitates H-abstraction. Nonlimiting examples of benzophenone derivatives include 4,4′-bis(dimethylamino)benzophenone, 4,4′-bis(carbazole)benzophenone, and 4,4′-bis(diphenylamino)-benzophenone. For further details regarding photoredox reactions performing H-abstraction using N-hydroxy-phthalimide and a benzophenone derivative, see Luo et al., “Aerobic oxidation of olefins and lignin model compounds using photogenerated phthalimide-N-oxyl radical,” Journal of Organic Chemistry 81: 9131-9137 (2016), the entire contents of which are incorporated by reference herein.

As provided herein, 5-mC and its derivatives may be modified in still other ways. For example, a photoredox reaction may be used to modify 5-mC or a derivative thereof, and a functional group then may be installed in the modified 5-mC or derivative thereof, which functional group may form a product such as described elsewhere herein. Illustratively, FIG. 5 schematically illustrates an example flow of operations in an alternative method for modifying methylcytosine or hydroxymethylcytosine using a photoredox reaction, and FIG. 6 schematically illustrates example structures formed using operations described with reference to FIG. 5. The 5-mC and/or 5-hmC may be present in a polynucleotide, and the polynucleotide may be present in an extracellular sample. Method 500 illustrated in FIG. 5 may include using a photoredox reaction to oxidize the 5-mC or 5-hmC to 5-fC (operation 510), for example as shown below to obtain 5-fC as illustrated at operation 610 of FIG. 6:

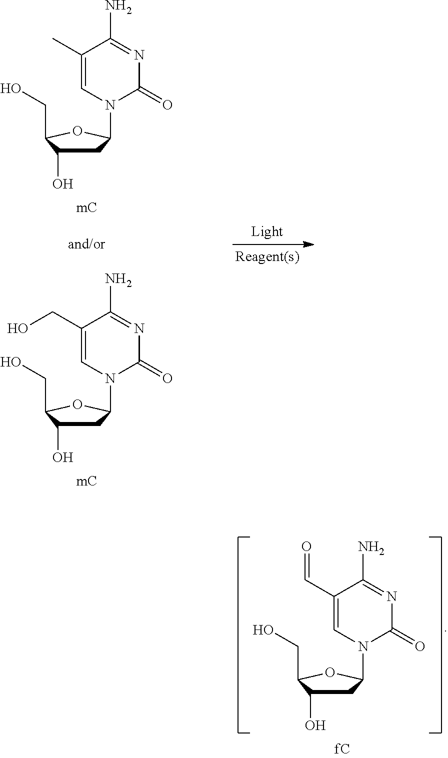

Nonlimiting examples of reagents that may be used to oxidize any 5-mC and/or 5-hmC in a polynucleotide to 5-fC include photocatalysts selected from the group consisting of: anthraquinone, 2,6-anthraquinone disulfonate, anthraquinone sulfonate, anthradione, menadione, naphtoquinone, methylene blue, rose Bengal, Eosin Y, thioxanthenone, 9-fluorenone, riboflavin, and flavin mononucleotide. The photocatalyst may be used in conjunction with an oxidant, which in some examples is selected from the group consisting of: dimethylsulfoxide, oxygen, hydrogen peroxide, air, and dialkysulfoxides. Example wavelengths are provided elsewhere herein. For further details regarding photocatalytic oxidation of 5-mC, see Jin et al., “A chemical photo-oxidation of 5-methyl cytidines,” Adv. Synth. Catal. 361: 4685-4690 (2019), the entire contents of which are incorporated by reference herein. For further details regarding photocatalytic oxidation of 2-aminobenzyl alcohols and secondary alcohols, see Xu et al., “Visible-light-mediated oxidative cyclization of 2-aminobenzyl alcohols and secondary alcohols enabled by an organic photocatalyst,” Journal of Organic Chemistry 86: 10747-10754 (2021), the entire contents of which are incorporated by reference herein.

Referring still to FIG. 5, method 500 may include installing a functional group at the 5-position of the 5-fC (operation 520). Optionally, the functional group may be installed using a photoredox reaction. Alternatively, the functional group may be installed through the use of a chemical reaction, without use of light. In some examples, the installed functional group modifies the 5-formyl group to provide functional group R₀ illustrated at operation 620 of FIG. 6, while in other examples the installed functional group replaces the 5-formyl group to provide functional group R₀ illustrated at operation 620. Nonlimiting examples of reactions and functional groups are provided further below. Method 500 also may include the installed functional group further reacting with the 5-fC to form a product having at least two rings (operation 530). Example structure of products resulting from operation 530, which products may be present in a polynucleotide, respectively are illustrated at operations 630 and 630′ in FIG. 2, and are shown further below as well:

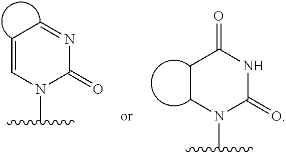 [SMILES: *n1cccnc1=O ; *N1CCC(=O)NC1=O]

The reaction of operation 520 may be performed subsequently to operation 510, or may be performed concurrently with operation 510. Additionally, or alternatively, the reaction of operation 530 may be performed subsequently to operation 520, or may be performed concurrently with operation 520. In some examples, operation 530 includes rearrangement of the intermediate generated using operation 520. Illustratively, the reaction may include an extra reagent (e.g., malononitrile), to carry out the cyclisation. So in this example there would be a single reaction mixture containing the photocatalyst, the oxidant and the cyclisation reagent.

In some examples, Friedländer cyclization is used to install the functional group at the 5-position of 5-fC. Nonlimiting examples of reagents that may be used include malononitrile, acetophenone, 3-oxobutyronitrile, and benzoylacetonitrile. For further details regarding use of Friedländer cyclization to install functional groups at the 5-position of 5-fC, see the following references, the entire contents of each of which are incorporated by reference herein: Zeng et al., “Bisulfite-free, nanoscale analysis of 5-hydroxymethylcytosine at single base resolution,” J. Am. Chem. Soc. 140: 13190-13194 (2018); Zhu et al., “Single-cell 5-formylcytosine landscapes of mammalian early embryos and ESCs at single-base resolution,” Cell Stem Cell 20: 720-731 (2017); and Ma et al., “One-pot intramolecular cyclization of 5-hydroxymethylcytosine for sequencing DNA hydroxymethylation at single-base resolution,” Analyst 146: 820-824 (2021). However, note that Zhu reports the Friedländer chemical cyclisation of fC (no oxidation, so the method can be used only to detect fC and not hmC or mC). Ma reports the Friedländer chemical cyclisation of hmC (not photochemical); moreover, this method uses harsh reagents including organic solvent, strong base and high temperature which could cause high degradation of DNA samples. Such degradation may be inhibited or prevented using the photochemical method provided herein. Zeng proposes a method for hmC detection using sequential chemical oxidation to fC with KRuO₄, followed by chemical Friedländer cyclisation. However, the KRuO₄ oxidation is damaging to DNA, and likely may not be carried out simultaneously with the cyclisation step as is an option in the present methods.

### Methods of Detecting Methylcytosine or Derivative Thereof in a Polynucleotide

In some examples, operations and structures such as described with reference to FIGS. 1-6 are used to detect methylation of DNA, including detection of 5-mC, 5-hmC, 5-fC, and/or 5-caC.

The TAPS (TET-assisted pyridine borane sequencing) workflow is described in the following references, the entire contents of each of which are incorporated by reference herein: Liu et al., “Bisulfite-free direct detection of 5-methylcytosine at base resolution,” Nature Biotechnology 37: 424-429 (2019); Liu et al., “Accurate targeted long-read DNA methylation and hydroxymethylation sequencing with TAPS,” Genome Biology 21: Article no. 54 (2020); Liu et al., “Subtraction-free and bisulfite-free specific sequencing of 5-methylcytosine and its oxidized derivatives at base resolution,” Nature Communications 12: Article no. 618 (2021); and International Publication No. WO 2019/136413 to Song et al. Briefly, and as described in these references, the TAPS workflow uses a ten-eleven translocation (TET) dioxygenase to oxidize any 5-mC, 5-hmC, and/or 5-fC in a polynucleotide to 5-caC.

In some examples provided herein, 5-caC (which may be prepared using an enzymatic reagent such as TET, or using one or more chemical reagents such as described elsewhere herein), is subjected to a photoredox reaction to obtain a product having a pattern of electron density which is sufficiently similar to that of thymine (T) to base pair with A and to be amplified as T during polymerase chain reaction (PCR). The product is sequenced as a T to detect locations where 5-mC, 5-hmC, or 5-fC had been located. In other examples provided herein, 5-mC is subjected to a photoredox reaction to obtain a product having a pattern of electron density which is sufficiently similar to that of thymine (T) to base pair with A and be amplified as T during polymerase chain reaction (PCR). The product is sequenced as a T to detect locations where 5-mC had been located. In still other examples provided herein, 5-mC and/or 5-hmC is subjected to a photoredox reaction to obtain 5-fC. The 5-fC then is reacted to obtain a product having a pattern of electron density which is sufficiently similar to that of thymine (T) to base pair with A and be amplified as T during polymerase chain reaction (PCR). The product is sequenced as a T to detect locations where 5-mC or 5-mC had been located.

The present inventors have recognized that the previously known TAPS workflow presents several challenges which may impede practical commercial implementation. For example, reduction of 5-carboxylcytosine using the pyridine borane complex requires a long incubation time (e.g., about 16 hours) at low pH, high temperature, and a high concentration of reagent (e.g., about 1 M) in order to be efficient. It is believed that these reaction conditions may cause considerable degradation of the DNA, reducing reaction yield and particularly degrading heavily methylated regions. Additionally, the pyridine borane complex is highly toxic and volatile, and requires the use of specialized equipment (such as a fume hood) which may not be compatible with automated sample preparation as may be desirable for use in a commercial implementation. The picoline borane complex also is believed not to be suitable for commercial implementation for similar reasons.

Without wishing to be bound by any theory, it is believed that by suitably selecting photoredox reaction(s) and reagent(s) with which 5-mC, 5-hmC, 5-fC, and/or 5-caC may react, the product of the reaction (including any rearrangements) may have a pattern of electron density which is sufficiently similar to that of T to be amplified as T during PCR. For example, FIG. 7A schematically illustrates example hydrogen bonding between 5-caC and guanine (G) in a double-stranded polynucleotide. In a manner such as illustrated in FIG. 7A, the exocyclic primary amine in position 4 of 5-caC acts as a hydrogen-bond donor by sharing its hydrogen with the keto group in position 6 of G, while the heterocyclic tertiary amine in position 3 and keto group in position 2 of 5-caC act as hydrogen-bond acceptors with which the secondary amine in position 1 and exocyclic primary amine in position 2 of G respectively share hydrogens. As illustrated in FIG. 2, installation of a functional group via a photoredox reaction (operation 220), and subsequent rearrangement to obtain product T* (operation 230, 230′) may convert the exocyclic, hydrogen-bond-donating primary amine in position 4 of 5-caC into an hydrogen-bond-accepting tertiary amine, and may convert the heterocyclic tertiary amine in position 3 of 5-caC into an hydrogen-bond-donating secondary amine. FIG. 7B schematically illustrates example hydrogen bonding between T* (caC modified using photoredox reaction and installation of functional group to form a bicyclic product in a manner such as provided herein), and adenosine in a double-stranded polynucleotide. In a manner such as illustrated in FIG. 7B, the heterocyclic secondary amine in position 3 of T* (modified cytosine) acts as an hydrogen-bond-donor that shares its hydrogen with the heterocyclic tertiary amine in position 1 of A, while the tertiary amine in the second ring of T* acts as an hydrogen-bond-acceptor with which the exocyclic primary amine in position 6 of A shares hydrogen. Accordingly, it may be understood that operation 230, 230's conversion of hydrogen-bond-donating primary amine in position 4 of 5-caC into the hydrogen-bond-accepting tertiary amine of T* yields a moiety that may not hydrogen bond with a corresponding moiety of G. Additionally, it may be understood that operation 230, 230's conversion of the heterocyclic tertiary amine in position 3 of 5-caC into the hydrogen-bond-donating secondary amine of T* yields a moiety that may share its hydrogen with the tertiary amine in position 1 of A. Such modifications facilitate T* preferentially binding to A, rather than with G as is the case of caC. Although not specifically illustrated in FIG. 7B, it will be appreciated that other bicyclic products obtained using 5-mC, 5-hmC, 5-fC, or 5-caC using the methods described herein may have similar patterns of electron donation and electron withdrawal, e.g., such as illustrated in FIGS. 4 and 6.

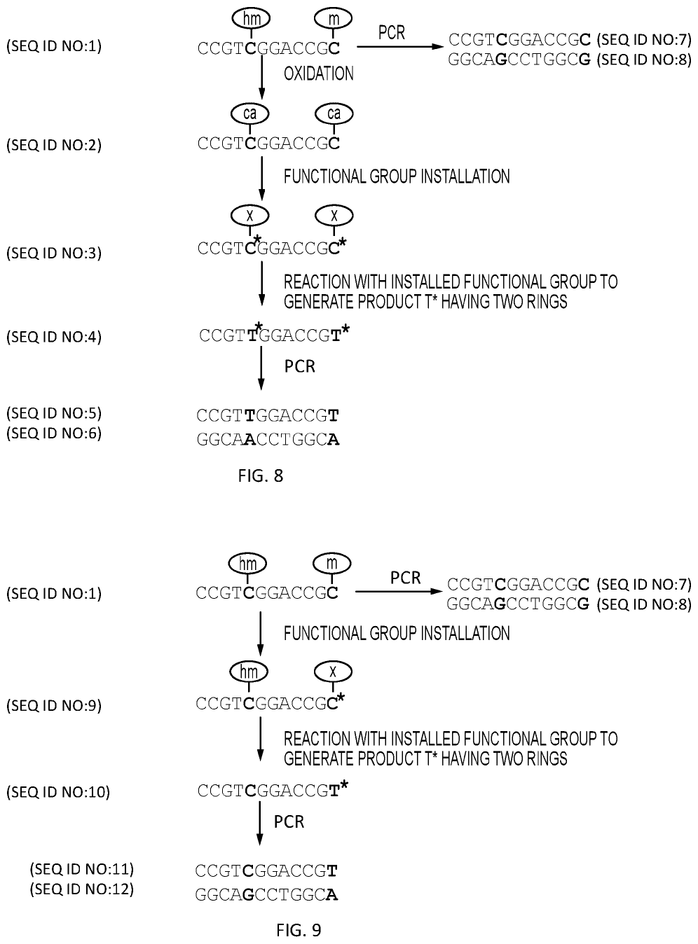

FIG. 8 schematically illustrates example operations for detecting methylcytosine or derivative thereof in a polynucleotide using operations, structures, and hydrogen bonding such as described with reference to FIGS. 1, 2, and 7A-7B. The workflow (method) illustrated in FIG. 8 includes oxidizing any 5-methylcytosine, 5-hydroxymethylcytosine, or 5-formylcytosine in the polynucleotide to 5-carboxylcytosine. Illustratively, in the nonlimiting example shown in FIG. 8, the polynucleotide has the sequence CCGThmCGGACCGmC (SEQ ID NO: 1), and TET dioxygenase or any suitable chemical reagent(s) is used to oxidize the hmC and mC to caC in a manner similar to that described in the above-cited references, yielding the sequence CCGTcaCGGACCGcaC (SEQ ID NO: 2). The polynucleotide then is contacted with suitable reagent(s) and light in a photoredox reaction, in a manner such as described further above with reference to FIGS. 1 and 2. The reaction product C* is represented in FIG. 8 as including moiety X corresponding to the installed functional group (group R₀ described with reference to operation 220 of FIG. 2). In the nonlimiting example illustrated in FIG. 8, the reaction product C* in the sequence CCGTC*GGACCGC* (SEQ ID NO: 3) spontaneously rearranges to T*, yielding the sequence CCGTT*GGACCGT* (SEQ ID NO: 4). Note that although such spontaneous rearrangement is illustrated as an operation of FIG. 8 for clarity, it should be appreciated that such rearrangement may occur spontaneously with installation of the functional group, as opposed to as a separate operation. As such, in FIG. 8, C* corresponds to a cytosine the 5′ position of which is coupled to a functional group X (corresponding to R₀ in FIG. 2) where the functional group is capable of reacting with the cytosine to form a product having two rings, and T* corresponds to the bicyclic product resulting from such reaction (230 or 230′ illustrated in FIG. 6) and having a hydrogen bonding pattern that is sufficiently similar to T as to pair with A.

The mC, hmC, and/or fC then may be detected using the T*. For example, as illustrated in FIG. 8, a first set of PCR reactions then may be performed on the product of the photoredox reaction and rearrangement(s) to generate amplicons of such product. In such amplicons, the T* (resulting from photoredox reaction(s) and rearrangement(s) such as described with reference to FIGS. 1 and 2) base pairs with A and is amplified as T, illustratively yielding the sequence 5′-CCGTTGGACCGT-3′ (SEQ ID NO: 5) (and complementary sequence 3′-GGCAACCTGGCA-5′ (SEQ ID NO: 6)). Additionally, a second set of PCR reactions may be performed on a separate aliquot of the unreacted polynucleotide. In such amplicons, the mC, hmC, and fC base pair with G and are amplified as C, illustratively yielding the sequence 5′-CCGTCGGACCGC-3′ (SEQ ID NO: 7) (and complementary sequence 3′-GGCAGCCTGGCG-5′ (SEQ ID NO: 8)). The locations in the target polynucleotide at which mC, hmC, or fC were located and at which T* was generated using the present operations, may be determined by comparing the sequence of the amplicons from the first set of PCR reactions to the sequence of amplicons from the second set of PCR reactions. Bases that are T (or A) in the amplicons from the first set of PCR reactions and that are C (or G) in the amplicons from the second set of PCR reactions may be identified as corresponding to mC, hmC, or fC because they were converted to T* using the present operations.

In another example, FIG. 9 schematically illustrates example operations for detecting methylcytosine in a polynucleotide using operations, structures, and hydrogen bonding such as described with reference to FIGS. 3 and 4. The workflow illustrated in FIG. 9 includes contacting the polynucleotide suitable reagent(s) and light in a photoredox reaction, in a manner such as described further above with reference to FIGS. 3 and 4. The reaction product C* is represented in FIG. 9 as including moiety X corresponding to the installed functional group (R₀ described with reference to operation 420 of FIG. 4). Note, however that the hmC in the sequence is not similarly reacted. In the nonlimiting example illustrated in FIG. 9, the reaction product C* in the sequence CCGThmCGGACCGC* (SEQ ID NO: 9) spontaneously rearranges to T*, yielding the sequence CCGThmCGGACCGT* (SEQ ID NO: 10). Note that although such spontaneous rearrangement is illustrated as an operation of FIG. 9 for clarity, it should be appreciated that such rearrangement may occur spontaneously with installation of the functional group, as opposed to as a separate operation. As such, in FIG. 9, C* corresponds to a cytosine the 5′ position of which is coupled to a functional group X (corresponding to R₀ in FIG. 4) where the functional group is capable of reacting with the cytosine to form a product having two rings, and T* corresponds to the bicyclic product resulting from such reaction (430 or 430′ illustrated in FIG. 4) and having a hydrogen bonding pattern that is sufficiently similar to T as to pair with A.

The mC then may be detected using the T*. For example, as illustrated in FIG. 9, a first set of PCR reactions then may be performed on the product of the photoredox reaction and rearrangement(s) to generate amplicons of such product. In such amplicons, the T* (resulting from photoredox reaction(s) and rearrangement(s) such as described with reference to FIGS. 3 and 4) base pairs with A and is amplified as T, illustratively yielding the sequence 5′-CCGTCGGACCGT-3′ (SEQ ID NO: 11) (and complementary sequence 3′-GGCAGCCTGGCA-5′ (SEQ ID NO: 12)). Additionally, a second set of PCR reactions may be performed on a separate aliquot of the unreacted polynucleotide. In such amplicons, the mC, hmC, and fC base pair with G and are amplified as C, illustratively yielding the sequence 5′-CCGTCGGACCGC-3′ (SEQ ID NO: 7) (and complementary sequence 3′-GGCAGCCTGGCG-5′ (SEQ ID NO: 8)). The locations in the target polynucleotide at which mC was located and at which T* was generated using the present operations, may be determined by comparing the sequence of the amplicons from the first set of PCR reactions to the sequence of amplicons from the second set of PCR reactions. Bases that are T (or A) in the amplicons from the first set of PCR reactions and that are C (or G) in the amplicons from the second set of PCR reactions may be identified as corresponding to mC because they were converted to T* using the present operations.

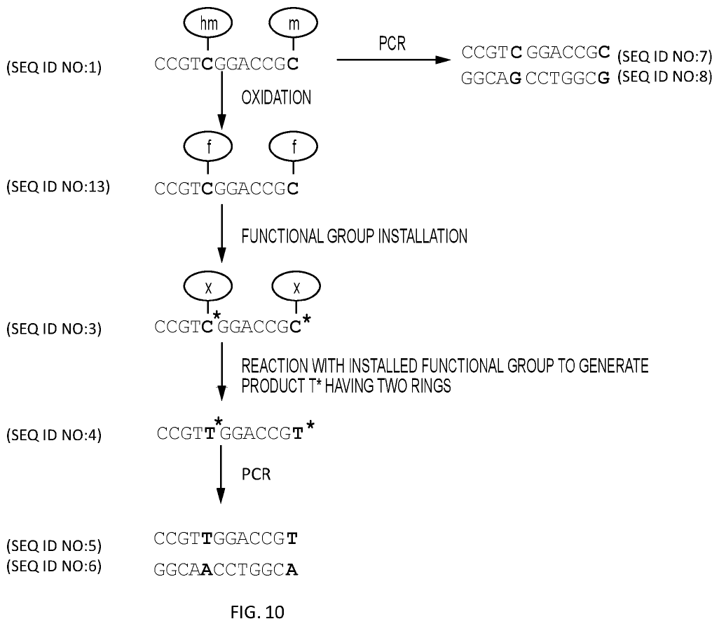

FIG. 10 schematically illustrates example operations for detecting methylcytosine or hydroxymethylcytosine in a polynucleotide using operations, structures, and hydrogen bonding such as described with reference to FIGS. 5 and 6. The workflow (method) illustrated in FIG. 10 includes oxidizing any 5-methylcytosine or 5-hydroxymethylcytosine in the polynucleotide to 5-formylcytosine. Illustratively, in the nonlimiting example shown in FIG. 10, the polynucleotide has the sequence CCGThmCGGACCGmC (SEQ ID NO: 1), and a photoredox reaction is used to oxidize the hmC and mC to fC in a manner such as described with reference to FIGS. 5 and 6, yielding the sequence CCGTfCGGACCGIC (SEQ ID NO: 13). The polynucleotide is contacted with suitable reagent(s) (and optionally light in a photoredox reaction), in a manner such as described further above with reference to FIGS. 5 and 6. The reaction product C* is represented in FIG. 10 as including moiety X corresponding to the installed functional group (R₀ described with reference to operation 620 of FIG. 6). Optionally, the functional group may be installed concurrently with oxidation of mC and hmC to fC. In the nonlimiting example illustrated in FIG. 10, the reaction product C* in the sequence CCGTC*GGACCGC* (SEQ ID NO: 3) spontaneously rearranges to T*, yielding the sequence CCGTT*GGACCGT* (SEQ ID NO: 4). Note that although such spontaneous rearrangement is illustrated as an operation of FIG. 10 for clarity, it should be appreciated that such rearrangement may occur spontaneously with installation of the functional group, as opposed to as a separate operation. As such, in FIG. 10, C* corresponds to a cytosine the 5′ position of which is coupled to a functional group X (corresponding to R₀ in FIG. 6) where the functional group is capable of reacting with the cytosine to form a product having two rings, and T* corresponds to the bicyclic product resulting from such reaction (630 or 630′ illustrated in FIG. 6) and having a hydrogen bonding pattern that is sufficiently similar to T as to pair with A.

The mC and hmC then may be detected using the T*. For example, as illustrated in FIG. 10, a first set of PCR reactions then may be performed on the product of the photoredox reaction and rearrangement(s) to generate amplicons of such product. In such amplicons, the T* (resulting from photoredox reaction(s) and rearrangement(s) such as described with reference to FIGS. 5 and 6) base pairs with A and is amplified as T, illustratively yielding the sequence 5′-CCGTTGGACCGT-3′ (SEQ ID NO: 5) (and complementary sequence 3′-GGCAACCTGGCA-5′ (SEQ ID NO: 6)). Additionally, a second set of PCR reactions may be performed on a separate aliquot of the unreacted polynucleotide. In such amplicons, the mC and hmC base pair with G and are amplified as C, illustratively yielding the sequence 5′-CCGTCGGACCGC-3′ (SEQ ID NO: 7) (and complementary sequence 3′-GGCAGCCTGGCG-5′ (SEQ ID NO: 8)). The locations in the target polynucleotide at which mC or hmC were located and at which T* was generated using the present operations, may be determined by comparing the sequence of the amplicons from the first set of PCR reactions to the sequence of amplicons from the second set of PCR reactions. Bases that are T (or A) in the amplicons from the first set of PCR reactions and that are C (or G) in the amplicons from the second set of PCR reactions may be identified as corresponding to mC or hmC because they were converted to T* using the present operations.

### Additional Comments

While various illustrative examples are described above, it will be apparent to one skilled in the art that various changes and modifications may be made therein without departing from the invention. The appended claims are intended to cover all such changes and modifications that fall within the true spirit and scope of the invention.

It is to be understood that any respective features/examples of each of the aspects of the disclosure as described herein may be implemented together in any appropriate combination, and that any features/examples from any one or more of these aspects may be implemented together with any of the features of the other aspect(s) as described herein in any appropriate combination to achieve the benefits as described herein.

1. A method of modifying 5-carboxylcytosine (5-caC) in a polynucleotide, the method comprising:
using a photoredox reaction to install a functional group at the 5-position of the 5-caC,
wherein the installed functional group further reacts with the 5-caC to form a product having at least two rings.

2. The method of claim 1, wherein the product has the structure:

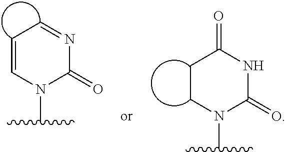 [SMILES: *n1cccnc1=O ; *N1CCC(=O)NC1=O]

3. The method of claim 1, wherein the photoredox reaction comprises a photoinduced decarboxylative radical reaction replacing the 5-carboxyl group with the functional group R₀:

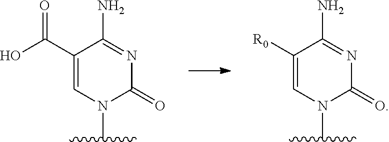

4. The method of claim 3, wherein the photoinduced decarboxylative radical reaction uses 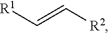
a photocatalyst or photosensitizer, and light.

5. The method of claim 4, wherein R¹ comprises an electron withdrawing group.

6. (canceled)

7. The method of claim 4, wherein R² promotes formation of at least one of the two rings.

8. The method of claim 4, wherein R¹ is an ester, and R² is H.

9. (canceled)

10. The method of claim 4, wherein R¹ is an amide, and R² is ethanethiol.

11. (canceled)

12. The method of claim 4, wherein: 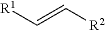
is diethyl ethylidenemalonate.

13. The method of claim 1, wherein the reaction further uses (i) a donor-acceptor complex or (ii) a copper salt and an oxidant.

14-20. (canceled)

21. The method of claim 20, wherein the photoredox reaction uses 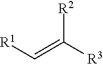
and light.

22. The method of claim 21, wherein R¹ is an electron withdrawing group or includes an aryl group.

23. (canceled)

24. The method of claim 21, wherein R² and R³ promote formation of at least one of the two rings.

25-32. (canceled)

33. A method of modifying 5-methylcytosine (5-mC) in a polynucleotide, the method comprising:
using a photoredox reaction to install a functional group at the 5-methyl group of the 5-mC,
wherein the installed functional group further reacts with the 5-mC to form a product having at least two rings.

34-41. (canceled)

42. A method of modifying 5-methylcytosine (5-mC), 5-hydroxymethylcytosine (5-hmC), or 5-formylcytosine (5-fC) in a polynucleotide, the method comprising:
oxidizing the 5-mC, 5-hmC, or 5-fC to 5-carboxylcytosine (5-caC); and
using the method of claim 1 to generate the product.

43. The method of claim 42, wherein a ten-eleven translocation (TET) dioxygenase is used to oxidize the 5-mC, 5-hmC, or 5-fC to 5-caC.

44. The method of claim 42, wherein oxidizing the 5-mC, 5-hmC, or 5-fC to 5-carboxylcytosine (5-caC) comprises contacting the 5-mC, 5-hmC, or 5-fC with one or more chemical reagents.

45. A method of detecting 5-methylcytosine (5-mC), 5-hydroxymethylcytosine (5-hmC), or 5-formylcytosine (5-fC) in a polynucleotide, the method comprising:
modifying the 5-mC, 5-hmC, or 5-fC using the method of claim 42 to generate a modified polynucleotide comprising the product; and
detecting the 5-mC, 5-hmC, or 5-fC using the modified polynucleotide.

46. A method of detecting 5-methylcytosine (5-mC) in a polynucleotide, the method comprising:
modifying the 5-mC using the method of claim 33 to generate a modified polynucleotide comprising the product; and
detecting the 5-mC using the modified polynucleotide.

47. A method of modifying 5-methylcytosine (5-mC) or 5-hydroxymethylcytosine (5-hmC), in a polynucleotide, the method comprising:
using a first photoredox reaction to oxidize the 5-mC or 5-hmC to 5-fC; and
installing a functional group at the 5-position of the 5-fC,
wherein the installed functional group further reacts with the 5-fC to form a product having at least two rings.

48-70. (canceled)


SEQUENCE LISTING (13 sequences)

SEQ ID NO: 1 - DNA, 12 bp, synthetic construct
  source 1..12: mol_type = other DNA
  modified_base 5: mod_base = OTHER; note = hydroxymethylcytosine
  modified_base 12: mod_base = OTHER; note = methylcytosine
ccgtcggaccgc

SEQ ID NO: 2 - DNA, 12 bp, synthetic construct
  source 1..12: mol_type = other DNA
  modified_base 5: mod_base = OTHER; note = carboxylcytosine
  modified_base 12: mod_base = OTHER; note = carboxylcytosine
ccgtcggaccgc

SEQ ID NO: 3 - DNA, 12 bp, synthetic construct
  source 1..12: mol_type = other DNA
  modified_base 5: mod_base = OTHER; note = a cytosine the 5 prime position of which is coupled to a functional group X where the functional group is capable of reacting with the cytosine to form a product having two rings
  modified_base 12: mod_base = OTHER; note = a cytosine the 5 prime position of which is coupled to a functional group X where the functional group is capable of reacting with the cytosine to form a product having two rings
ccgtcggaccgc

SEQ ID NO: 4 - DNA, 12 bp, synthetic construct
  source 1..12: mol_type = other DNA
  modified_base 5: mod_base = OTHER; note = a bicyclic product having a hydrogen bonding pattern that is sufficiently similar to T as to pair with A, formed from cytosine reacting with functional group X
  modified_base 12: mod_base = OTHER; note = a bicyclic product having a hydrogen bonding pattern that is sufficiently similar to T as to pair with A, formed from cytosine reacting with functional group X
ccgttggaccgt

SEQ ID NO: 5 - DNA, 12 bp, synthetic construct
  source 1..12: mol_type = other DNA
ccgttggaccgt

SEQ ID NO: 6 - DNA, 12 bp, synthetic construct
  source 1..12: mol_type = other DNA
acggtccaacgg

SEQ ID NO: 7 - DNA, 12 bp, synthetic construct
  source 1..12: mol_type = other DNA
ccgtcggaccgc

SEQ ID NO: 8 - DNA, 12 bp, synthetic construct
  source 1..12: mol_type = other DNA
gcggtccgacgg

SEQ ID NO: 9 - DNA, 12 bp, synthetic construct
  source 1..12: mol_type = other DNA
  modified_base 5: mod_base = OTHER; note = hydroxymethylcytosine
  modified_base 12: mod_base = OTHER; note = a cytosine the 5 prime position of which is coupled to a functional group X where the functional group is capable of reacting with the cytosine to form a product having two rings
ccgtcggaccgc

SEQ ID NO: 10 - DNA, 12 bp, synthetic construct
  source 1..12: mol_type = other DNA
  modified_base 5: mod_base = OTHER; note = hydroxymethylcytosine
  modified_base 12: mod_base = OTHER; note = a bicyclic product having a hydrogen bonding pattern that is sufficiently similar to T as to pair with A, formed from cytosine reacting with functional group X
ccgtcggaccgt

SEQ ID NO: 11 - DNA, 12 bp, synthetic construct
  source 1..12: mol_type = other DNA
ccgtcggaccgt

SEQ ID NO: 12 - DNA, 12 bp, synthetic construct
  source 1..12: mol_type = other DNA
acggtccgacgg

SEQ ID NO: 13 - DNA, 12 bp, synthetic construct
  source 1..12: mol_type = other DNA
  modified_base 5: mod_base = OTHER; note = formylcytosine
  modified_base 12: mod_base = OTHER; note = formylcytosine
ccgtcggaccgc